(19) United States
(12) Patent Application Publication
(10) Pub. No.: US 2026/0041938 A1
(43) Pub. Date: Feb. 12, 2026

(54) USE OF LOW-INTENSITY PULSED ULTRASOUND (LIPUS) IN PREPARATION OF PRODUCT FOR TREATING DRY AGE-RELATED MACULAR DEGENERATION (dAMD)

(71) Applicant: TIANJIN MEDICAL UNIVERSITY EYE HOSPITAL, Tianjin (CN)

(72) Inventors: Yan SHAO, Tianjin (CN); Chenguang ZHENG, Tianjin (CN); Guanghua HOU, Tianjin (CN); Yunzhu KONG, Tianjin (CN); Ling WANG, Tianjin (CN); Ganjiang WEI, Tianjin (CN)

(21) Appl. No.: 18/989,304

(22) Filed: Dec. 20, 2024

(30) Foreign Application Priority Data
Aug. 8, 2024 (CN) 202411082538.1

(51) Int. Cl. A61N 7/00

(52) U.S. Cl. CPC A61N 7/00

(57) ABSTRACT

Provided is use of low-intensity pulsed ultrasound (LIPUS) in preparation of a product for treating dry age-related macular degeneration (dAMD), which belongs to the technical field of biomedicine. The present disclosure proposes for the first time that LIPUS can be used to treat the eye disease of dAMD. LIPUS allows for treating dAMD by transmitting acoustic energy to a target tissue. The LIPUS method provided by the present disclosure has the characteristics of non-invasiveness, painlessness, and no side effects.

### CROSS REFERENCE TO RELATED APPLICATION

This patent application claims the benefit and priority of Chinese Patent Application No. 2024110825381, filed with the China National Intellectual Property Administration on Aug. 8, 2024, the disclosure of which is incorporated by reference herein in its entirety as part of the present application.

### TECHNICAL FIELD

The present disclosure belongs to the technical field of biomedicine, and in particular relates to use of low-intensity pulsed ultrasound (LIPUS) in preparation of a product for treating dry age-related macular degeneration (dAMD).

### BACKGROUND

Age-related macular degeneration (AMD) is a common degenerative retinal disease that affects central vision, primarily in individuals at an age of 50 years or more. It can present in both eyes successively or simultaneously and leads to progressive visual impairment, which seriously affects the life quality of the elderly. AMD is divided into dry AMD and wet AMD according to clinical manifestations and pathological changes. Dry AMD (dAMD) has a gradual onset, with patients undergoing an imperceptible eyesight decline, which may also include metamorphopsia, typically affecting both eyes similarly. At an early stage of dAMD, yellowish-white round-like drusen of different sizes can be seen in the posterior pole of the fundus. They may fuse, accompanied by retinal pigment epithelium (RPE) proliferation or atrophy, loss of foveal reflex, and pigmentary disturbance in the posterior pole, eventually leading to clearly-defined geographic atrophy. At an advanced stage of dAMD, atrophy of the choriocapillaris may occur, exposing larger choroidal vessels. There is currently no effective treatment for dAMD. The existing methods for treating dAMD include intravitreal injection of drugs such as an anti-inflammatory complement, neuroprotective therapies, stem cell therapies, or the like, all of which tend to be invasive and have a high incidence of adverse events. Oral administration of antioxidants or an eye drops has a long treatment cycle and works slowly.

Low-intensity pulsed ultrasound (LIPUS) is a specialized type of ultrasound that transmits energy at a low intensity and outputs in the form of pulsed waves. LIPUS is an ultrasound with a special frequency usually of 1 MHz to 3 MHz and a low intensity usually of 20 mW/cm² to 200 mW/cm². It allows the transmission of acoustic energy to a target tissue with a minimal thermal effect, which enables a non-invasive physical stimulation for a therapeutic application. LIPUS has a variety of therapeutic applications. Studies have shown that LIPUS plays a role in the inhibition on inflammatory responses and neuroregulation, and can also promote the cartilage generation and repair of mesenchymal stem cells by regulating autophagy and enhance a therapeutic effect of mesenchymal stem cells in cartilage repair for osteoarthritis by regulating autophagy-mediated exosome release. Currently, there are no studies on the use of LIPUS in the treatment of dAMD.

### SUMMARY

In view of this, the present disclosure provides use of LIPUS in preparation of a product for treating dAMD. LIPUS allows for treating dAMD by transmitting acoustic energy to a target tissue, and is a non-invasive physical stimulation method.

To allow the above objective, the present disclosure provides the following technical solution.

The present disclosure provides use of LIPUS in preparation of a product for treating dAMD.

In some embodiments, the LIPUS has an intensity of 50 mW/cm² to 100 mW/cm² for treatment.

In some embodiments, the LIPUS has a duration of 10 min to 20 min for treatment.

In some embodiments, a stimulus-pulse duration is 0.5 s, an inter-stimulus interval is 2 s, and a center frequency is 0.5 MHz.

In some embodiments, before a treatment with the LIPUS, a medical coupling agent is applied, and then a LIPUS treatment probe is allowed to contact with an eyeball surface for ultrasound treatment.

In some embodiments, the product includes a device.

Embodiments of the present disclosure have the following beneficial effects.

The present disclosure proposes for the first time that LIPUS can be used to treat the eye disease of dAMD, and LIPUS can allow the purpose of treating dAMD by transmitting acoustic energy to a target tissue. The LIPUS method provided by the present disclosure has the characteristics of non-invasiveness, painlessness, and no side effects.

### BRIEF DESCRIPTION OF THE DRAWINGS

FIG. 1 is a schematic diagram of the treatment;

FIGS. 2A-2D show comparison of retina thicknesses in different groups, where FIG. 2A shows results of the control group; FIG. 2B shows results of the SI group; FIG. 2C shows results of the SI+U group; in FIG. 2A, FIG. 2B, and FIG. 2C, the left panels show locations to measure the retina thickness and the right panels show schematic diagrams of retina thicknesses at the corresponding positions; and FIG. 2D shows statistical results of retina thicknesses, where * indicates p<0.05, ** indicates p<0.01, and **** indicates p<0.0001;

FIGS. 3A-3E show comparison of the electroretinograms (ERGs) in different groups, where FIG. 3A shows results of the control group; FIG. 3B shows results of the SI group; FIG. 3C shows results of the SI+U group; FIG. 3D shows statistics of the B-wave amplitude; FIG. 3E shows statistics of the A-wave amplitude; and ns indicates no statistical difference, * indicates p<0.05, ** indicates p<0.01, and **** indicates p<0.0001;

FIGS. 4A-4F show fundus images and hematoxylin and eosin (HE) staining results of different groups, where FIG. 4A shows a fundus image of the control group; FIG. 4B shows a fundus image of the SI group; FIG. 4C shows a fundus image of the SI+U group; FIG. 4D shows an HE staining result of the control group; FIG. 4E shows an HE staining result of the SI group; FIG. 4F shows an HE staining result of the SI+U group; and the red arrow indicates the pigment accumulation at a retinal pigment epithelium (RPE);

FIGS. 5A-5B show deposition of lipofuscin (red arrow) in different groups, where the left panel shows the deposition of lipofuscin in different groups; the right panel shows the statistics of the deposition; and ** indicates p<0.01 and *** indicates p<0.001;

FIGS. 6A-6B show detection results of cell apoptosis in different groups, where the cell apoptosis is represented by green fluorescence; FIG. 6A shows the cell apoptosis in different groups; FIG. 6B shows the statistics of cell apoptosis; and * indicates p<0.05, ** indicates p<0.01, and **** indicates p<0.0001;

FIGS. 7A-7C show detection results of cell senescence in different groups, where the cell senescence is represented by blue particles; FIG. 7A shows a result of the control group; FIG. 7B shows a result of the SI group; and FIG. 7C shows a result of the SI+U group; and

FIGS. 8A-8H show in vitro detection results of oxidative stresses (reactive oxygen species (ROS) levels) and cell senescence in different groups, where FIG. 8A shows an ROS level of the control group; FIG. 8B shows an ROS level of the SI group; FIG. 8C shows an ROS level of the SI+U group; FIG. 8D shows the statistics of ROS levels in different groups; FIG. 8E shows the cell senescence of the control group; FIG. 8F shows the cell senescence of the SI group; FIG. 8G shows the cell senescence of the SI+U group; FIG. 8H shows the statistical results of cell senescence levels in different groups; the green fluorescence indicates an ROS level and the blue indicates the cell senescence; and ns indicates no statistical difference, * indicates p<0.05, and ** indicates p<0.01.

### DETAILED DESCRIPTION OF THE EMBODIMENTS

The present disclosure provides use of LIPUS in preparation of a product for treating dAMD.

In the present disclosure, the LIPUS has a intensity of preferably 50 mW/cm² to 100 mW/cm² and more preferably 60 mW/cm² to 90 mW/cm², and a duration of preferably 10 min to 20 min and more preferably 12 min to 18 min for treatment. The duration is preferably for each treatment, and the treatment is preferably conducted once a day. In the present disclosure, when a LIPUS device is used to treat dAMD, parameters of the device are preferably as follows: stimulus-pulse duration (sd)=0.5 s, inter-stimulus interval (isi)=2 s, fundamental frequency (ff)=0.5 MHz, pulse repetition frequency (prf)=1.5 KHz, pulse number=750, duty cycle (dc)=20%, tone-burst duration tbd=0.1 ms, and burst cycle number (N)=50. The intensity includes two indicators: spatial-peak temporal-averaged intensity (Ispta)=99.6 mW/cm² (which can be directly equal to about 100) and peak pressure (pp)=0.282 Mpa.

The LIPUS defined by the present disclosure has the characteristics of strong permeation, deep penetrating, and uniform energy distribution. The treatment principle of the LIPUS adopted in the present disclosure mainly includes the following two aspects: a mechanical effect and a biological effect. The mechanical effect means that small pressure fluctuations are generated when acoustic vibrations of ultrasounds act on the retinal tissue. These pressure fluctuations can cause small vibrations of cell membranes to facilitate the inter- and extra cellular substance exchanges and increase the cell viability. In addition, ultrasounds can alter the fluid flow of the extracellular matrix, increase the delivery of nutrients and oxygen, and promote the metabolic activities of retinal cells. The biological effect means that acoustic vibrations of ultrasounds affect biological responses of retinal cells. The vibration of ultrasounds stimulates the signaling pathways in cells, and promotes the proliferation and differentiation of cells. In addition, ultrasounds can promote the synthesis and secretion of some important biological factors by retinal cells, regulate the apoptosis and inflammatory responses of cells, and delay the progression of dAMD. The LIPUS treatment provided by the present disclosure covers a wider range during the treating process compared to micropulsed low-energy laser treatment. The entire ultrasound probe contacts with the corneal surface, delivering pulsed waves that reach the whole retina, such that a corresponding treatment has a wide range and is safe and effective.

In the present disclosure, before a treatment with the LIPUS, preferably, a medical coupling agent needs to be applied, and then a LIPUS treatment probe is allowed to contact a surface of an eyeball for ultrasound treatment. A schematic diagram of the treatment is shown in FIG. 1. In the present disclosure, the product preferably includes a device.

The technical solutions provided by the present disclosure are described in detail below with reference to examples, but the examples cannot be understood as limiting the protection scope of the present disclosure.

Unless otherwise specified, all methods in the following examples are conventional methods.

All materials and reagents used in the following examples may be commercially available, unless otherwise specified.

### Example 1

Thirty-six male C57BL/6J mice that were 6 to 8 weeks old were randomly divided into a control group (control), a disease group (SI), and an ultrasound treatment group (SI+U). Except for mice in the control group (normal saline was injected intraperitoneally), all mice were intraperitoneally injected with a 30 mg/kg sodium iodate solution (which was prepared by dissolving a sodium iodate powder in normal saline) for modeling to construct an dAMD animal model. The LIPUS treatment was applied to mice in the ultrasound treatment group. A specific treatment method was as follows: a medical coupling agent was applied around eyes of mice, and a LIPUS treatment probe was allowed to contact with the eyeball surface for ultrasound treatment, where the intensity for treatment was 100 mW/cm², and the treatment was conducted once a day and lasted for 7 d, with a duration of 10 min each time. The remaining parameters of the LIPUS device were set as follows: stimulus-pulse duration (sd)=0.5 s, inter-stimulus interval (isi)=2 s, fundamental frequency (ff)=0.5 MHz, pulse repetition frequency (prf)=1.5 KHz, pulse number=750, duty cycle (dc)=20%, tone-burst duration (tbd)=0.1 ms, burst cycle number (N)=50, and peak pressure (pp)=0.282 Mpa.

At the end of the 7 d treatment, a biopsy was conducted for mice, then eyeballs were taken from the mice, and then some tests were conducted:

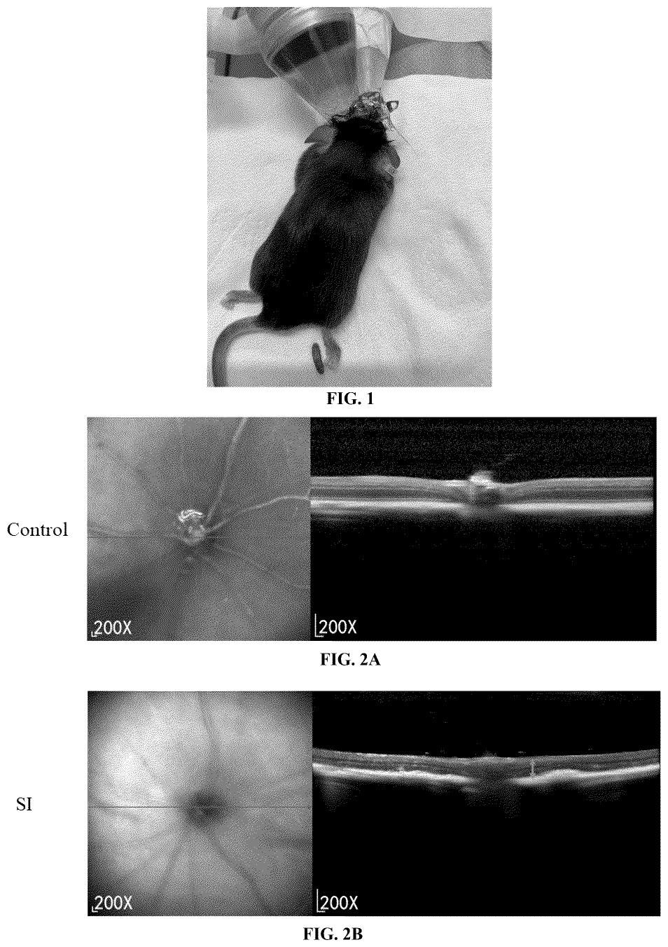

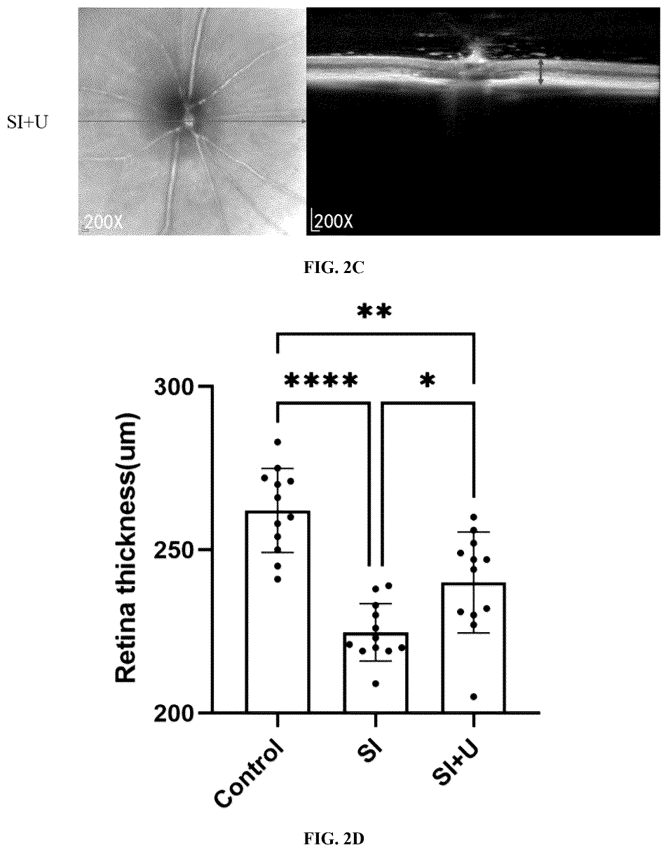

(1) Optical coherence tomography (OCT) was used to measure retina thicknesses in different groups, and final results were expressed as mean±standard deviation. Mice each were anesthetized intraperitoneally with 1.2% 2,2,2-tribromoethanol (0.2 mL/10 g). If the anesthesia time needed to be extended, 0.3 mL of anesthetic could be injected intraperitoneally before waking of mice. After anesthesia, the mydriasis was conducted with compound tropicamide. After the pupils were completely dilated, a gatifloxacin ophthalmic gel was applied to eyes to keep eyeballs moist and prevent the corneal edema, and images were taken and saved with an OCT machine. Results are shown in FIGS. 2A-2D. Compared with the control group, the SI group had tortuous and thinned retinas, and the retina thickness of the ultrasound treatment group was significantly improved compared to that of the disease group. The results indicate that the LIPUS treatment has a positive effect for dAMD.

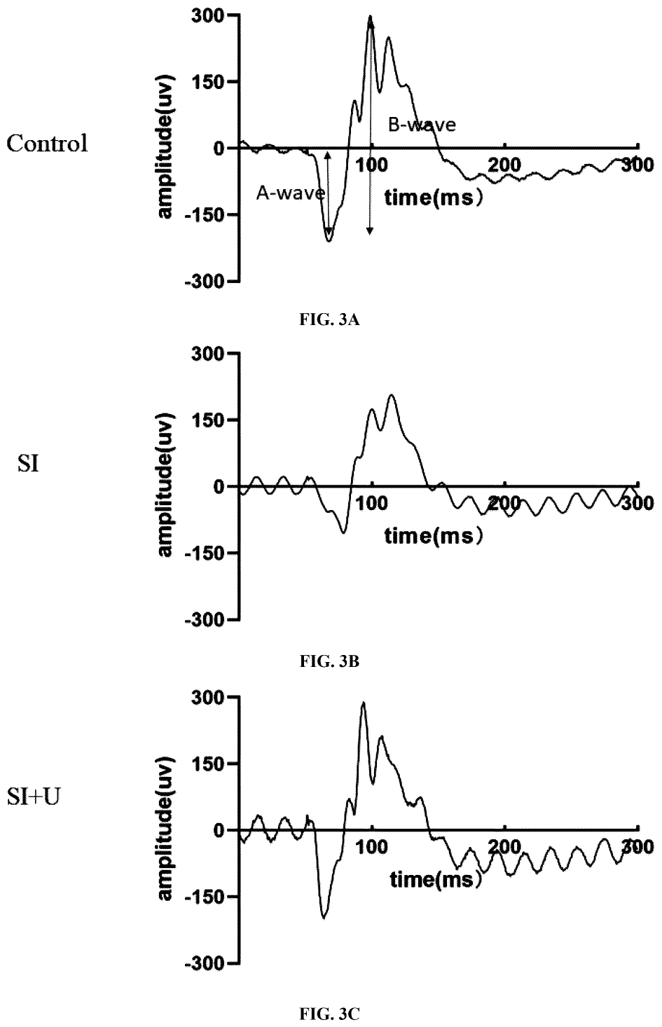

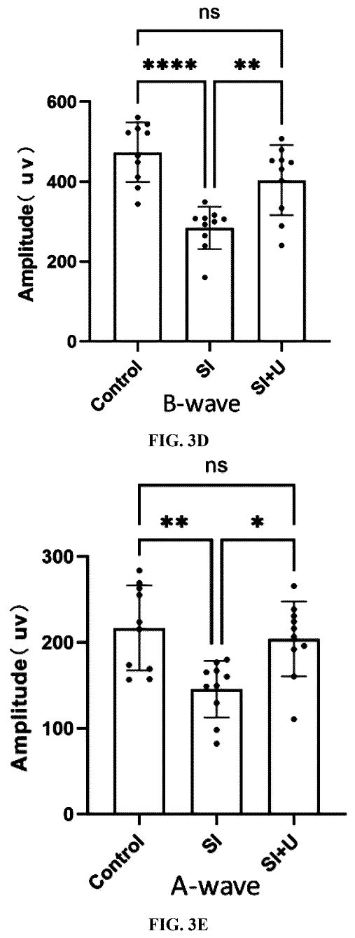

(2) ERGs were compared. ERG mainly reflects changes in retinal function, where the A-wave mainly reflects changes in the outer retinal function and B-wave mainly reflects changes in the inner retinal function. Mice were subjected to dark adaptation in a completely-dark room for 8 h. Then, under the illumination of a red-light flashlight, mice were subjected to anesthesia, mydriasis, gatifloxacin ophthalmic gel application for anti-inflammation (as described in the above step), and proparacaine ophthalmic anesthesia. Recording was conducted according to the instructions of the ERG machine. Eyes of mice were stimulated at a flash intensity of 2.2 cds/m², and changes in the retinal function were observed and recorded. The amplitude of the A-wave was measured from the baseline to the A-wave trough, and the amplitude of the B-wave was measured from the A-wave trough to the B-wave peak. The results are shown in FIGS. 3A-3E. Compared with the control group, SI group showed a decrease in both the A-wave amplitude and the B-wave amplitude. After the LIPUS treatment, the amplitudes increased to some degree, with statistically-significant differences. There was no significant difference between the treatment group and the control group.

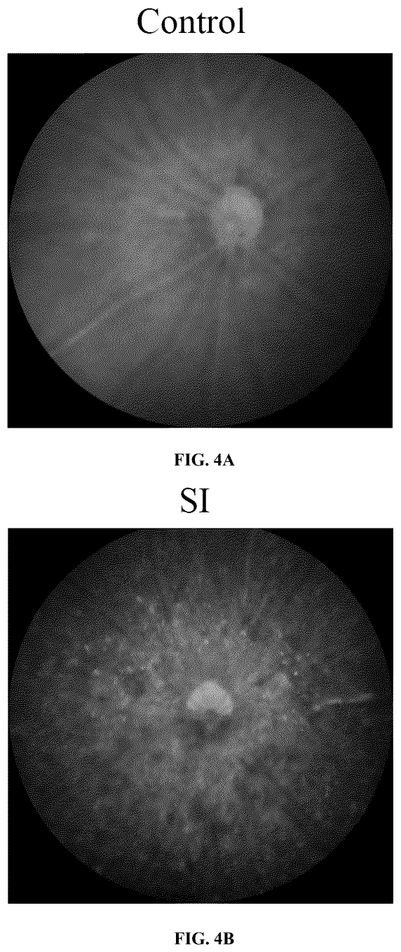

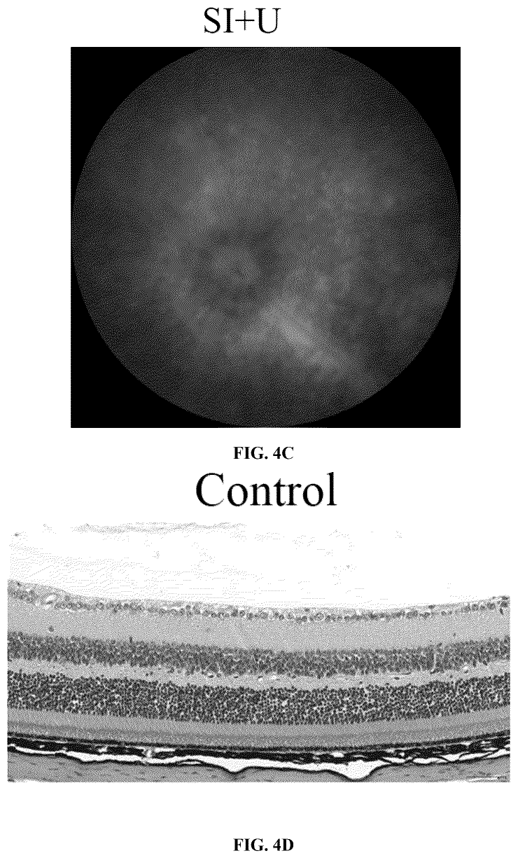

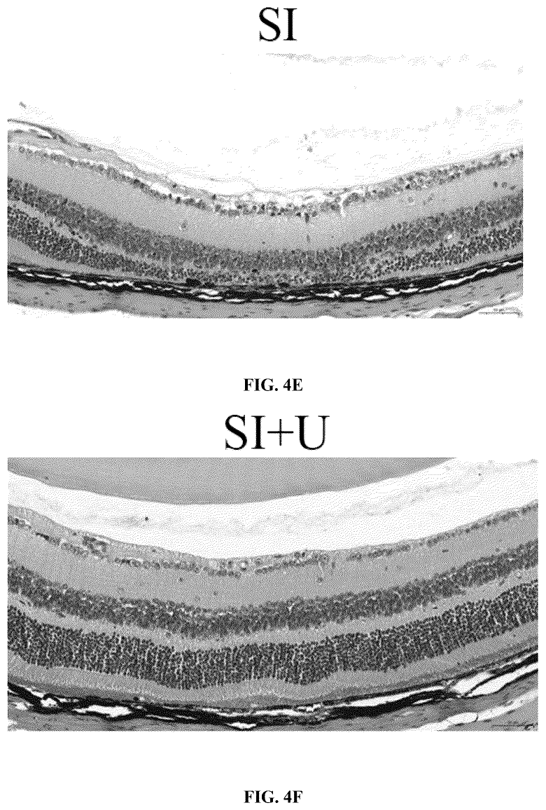

(3) Fundus images of different groups were observed. After anesthesia (the anesthesia operation was the same as above), the eye of a mouse was aligned with an fundus imager. Once the optic disc of the mouse was centered on the display, focus and brightness were adjusted, and an image was taken and saved by the fundus imager. The results are shown in FIG. 4A to FIG. 4C, where the disease group exhibited a significant amount of deposits in the fundus, which were reduced after LIPUS treatment.

Eyeballs of different groups each were embedded in a paraffin, cut into a 5 μm-thick section, and stained with HE, and changes in the retinal structure were observed and photographed under a microscope and compared. The results are shown in FIG. 4D to FIG. 4F. In the disease group, there was pigment accumulation (red arrow) at RPE and a structural disorder, and retinas were obviously thinned. In the LIPUS treatment group, retinas were not thinned, but the retinal structure was slightly tortuous compared with that of the control group.

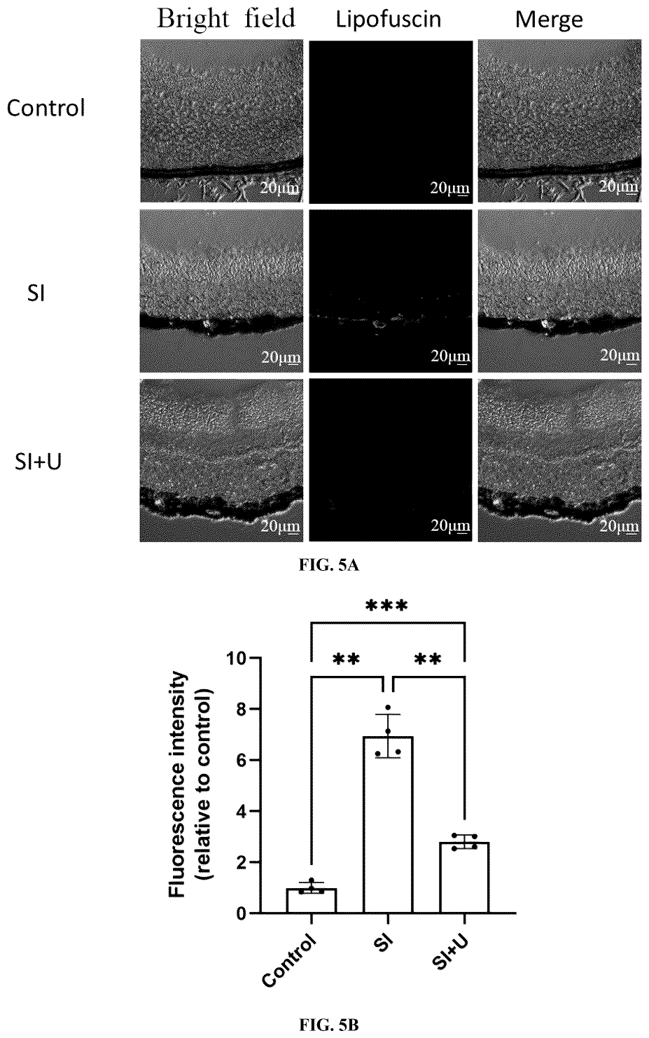

(4) Lipofuscin is a typical deposit of dAMD. The deposition of lipofuscin in different groups was detected. Frozen mouse eyeballs each were cut into an 8 μm-thick section, and the autofluorescence of lipofuscin was observed and recorded under a confocal microscope. The fluorescence intensity of each group was analyzed using the image J software. The results are shown in FIGS. 5A-5B, where the red arrow indicates the deposition of lipofuscin. The deposition of lipofuscin in the disease group was obvious. The deposition of lipofuscin in the treatment group was significantly reduced compared with that of the disease group, with a statistically-significant difference.

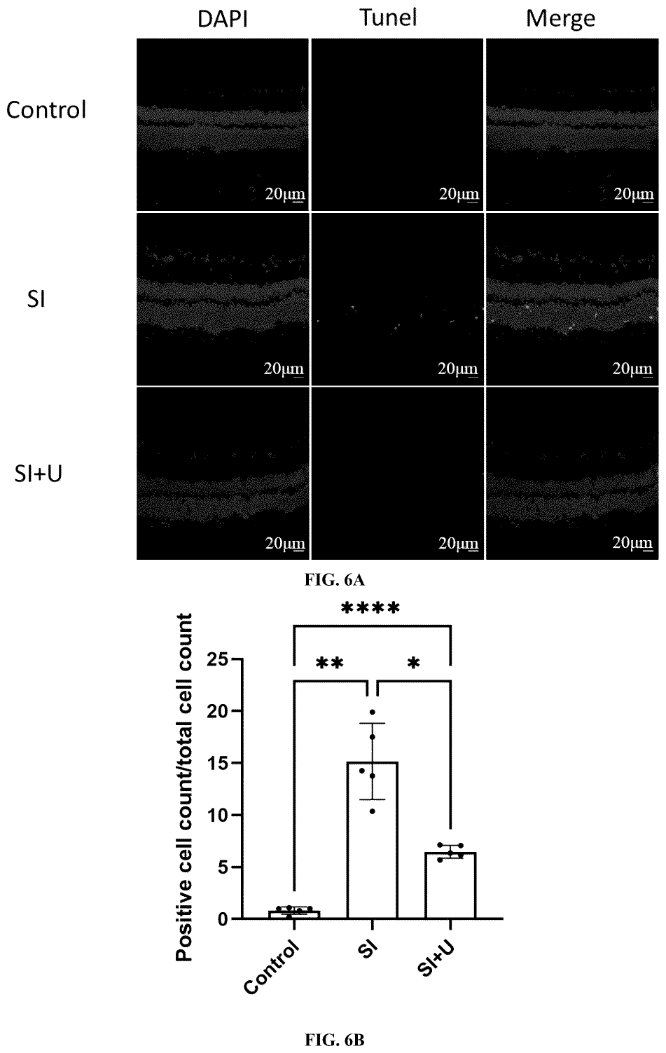

(5) The cell apoptosis (green fluorescence) in different groups was detected. According to the manufacturer′ instruction of the one-step TUNEL apoptosis assay kit (Beyotime, #C1088, Shanghai, China), the retinal section was subjected to apoptosis assay. An 8 μm-thick frozen section was re-warmed, fixed with a 4% histiocyte fixing solution for 40 min, permeabilized with 0.3% Triton X-100, then incubated with a TUNEL assay solution for 1 h in a 37° C. humid atmosphere, washed, subjected to nucleus counterstaining with DAPI, and finally mounted with an antifade mounting medium. Tunel-positive cells were observed under a confocal microscope, and results were expressed as the ratio of apoptotic cells to total cells. The results are shown in FIGS. 6A-6B. The number of apoptotic cells significantly increased in the disease group and significantly decreased in the treatment group, with a statistically-significant difference, indicating that the LIPUS treatment exhibited a positive effect for dAMD.

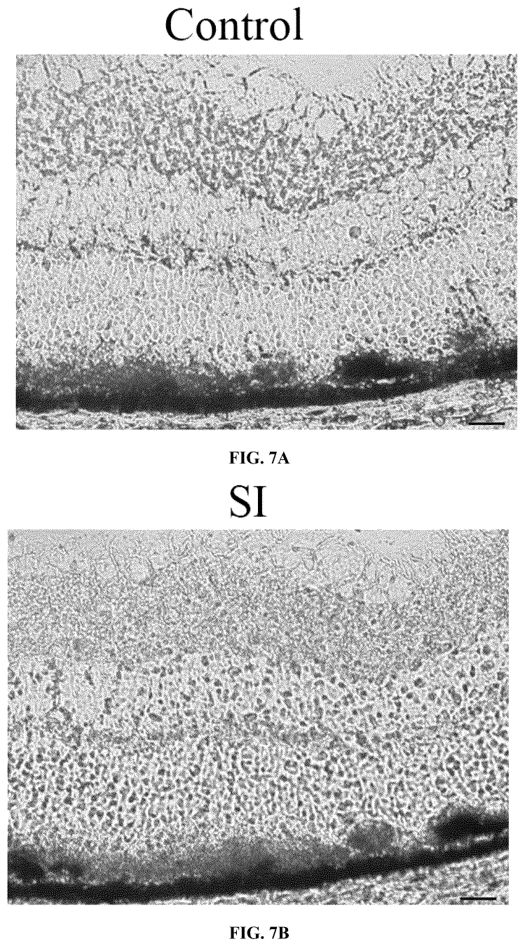

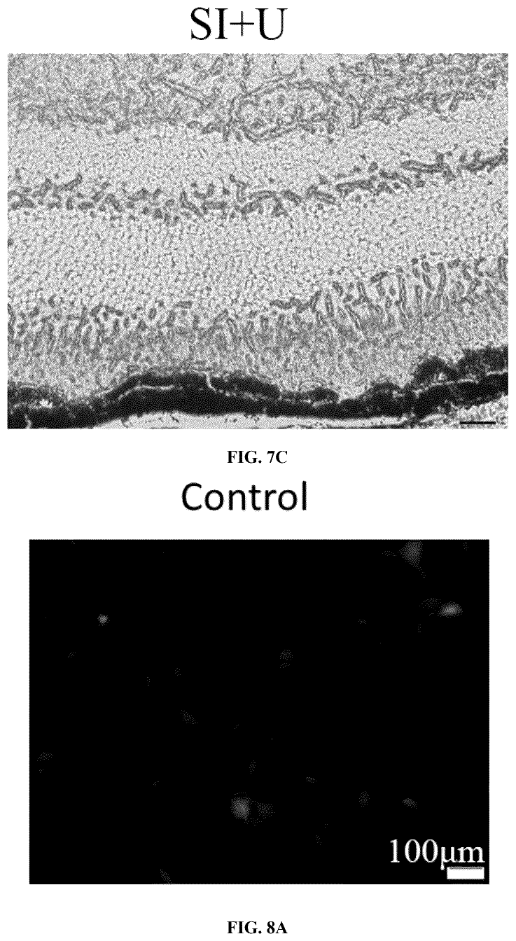

(6) The senescence of retinal tissues in different groups was detected. The frozen retinal section was subjected to senescence assay with a β-galactosidase in-situ staining kit (Beyotime, #RG0039, Shanghai, China). The frozen section was washed in PBS, fixed with a 4% histiocyte fixing solution for 15 min, washed to remove the fixing solution, incubated with a B-galactosidase staining working solution for 2 h in a 37° C. environment, washed with PBS to remove the staining working solution, and observed under a common optical microscope. The ratio of positive cells to total cells was analyzed using the image J software. The results are shown in FIGS. 7A-7C, where the blue particles indicated the staining results for cell senescence. The results showed that there were significant senescent positive cells (blue) in the disease group and the senescence was improved after treatment.

### Example 2

### In Vitro Detection of Oxidative Stresses (ROS Levels) and Senescence of RPE Cells

RPE cells were cultivated in vitro in a DMEM/F-12 (1:1) medium, and randomly divided into a control group (control), a disease group (SI), and an ultrasound treatment group (SI+U). Except for the control group, sodium iodate was added to a medium (a sodium iodate powder was dissolved in the medium, with a final concentration of 1 mM) in the disease group and the ultrasound treatment group, and 1 h later, a treatment was conducted. The LIPUS treatment was applied in the ultrasound treatment group. A specific treatment method involved applying low-intensity pulsed ultrasound to the bottom of the culture dish, with a treatment intensity of 50 mW/cm² for a duration of 20 minutes and a single treatment session. The remaining parameters of an LIPUS device were set as follows: sd=0.5 s, isi=2 s, ff=0.5 MHz, prf=1.5 KHz, a pulse number=750, dc=20%, tbd=0.1 ms, N=50, and pp=0.282 Mpa.

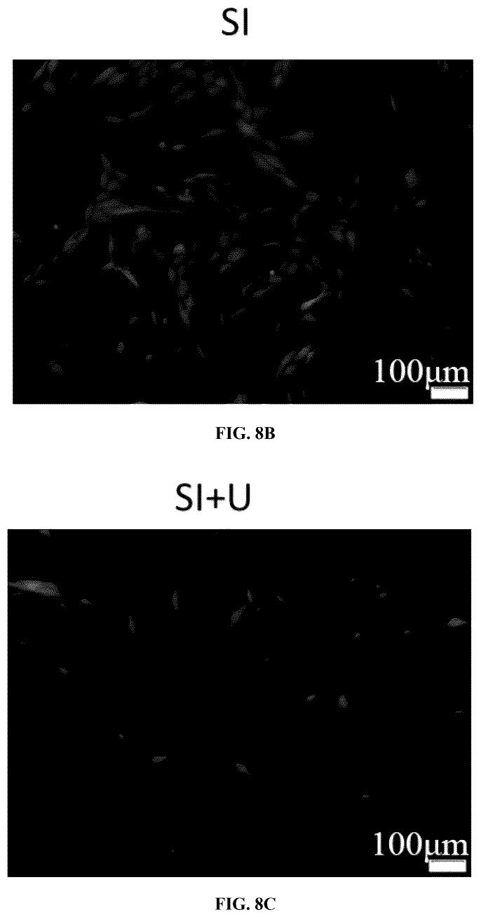

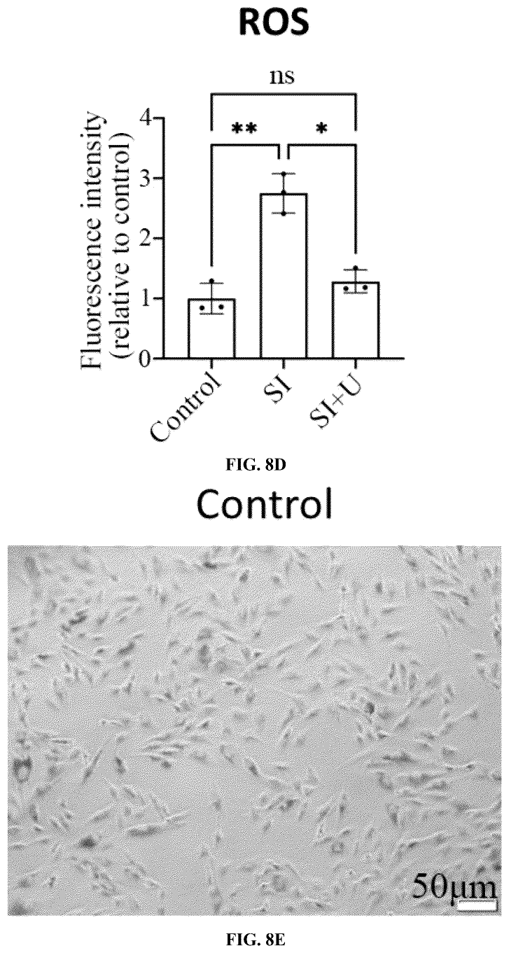

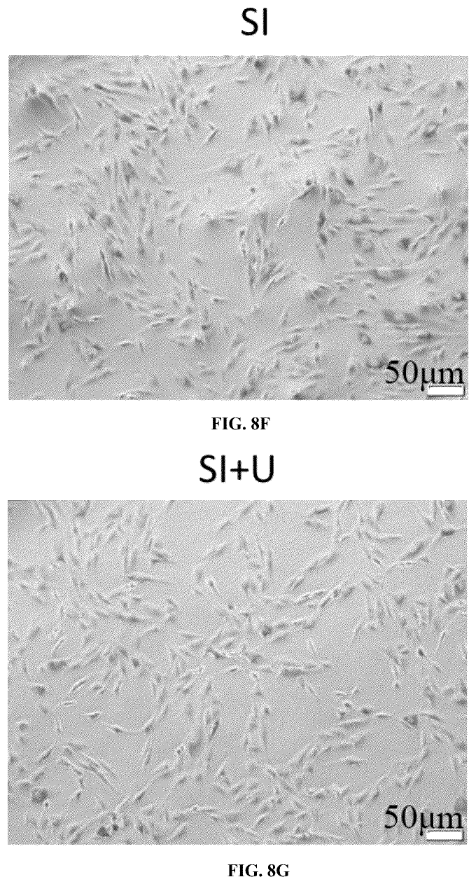

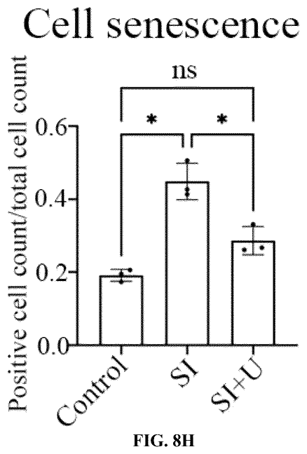

One day after sodium iodate was added, the ROS level of RPE cells was measured with an ROS assay kit (Beyotime, #S0033S, Shanghai, China). The cell culture medium was removed, then an ROS detection reagent was added, and cells were incubated in a 37° C. incubator for 0.5 h. The cells were washed three times with a serum-free cell culture medium to fully remove the reagent that had not entered the cells, and then the cells were observed and recorded under a fluorescence microscope. An average fluorescence intensity was analyzed using the image J software. The cell senescence detection steps were the same as the retinal tissue senescence detection steps above. The results are shown in FIGS. 8A-8H. In FIGS. 8A-8H, the green fluorescence indicated ROS and the blue indicated cell senescence. The results showed that the ROS (green fluorescence) intensity in the disease group was high, and after the treatment, the average fluorescence intensity was significantly reduced, with a statistically-significant difference. The cell senescence also exhibited the same trend, and after the treatment, the cell senescence level was significantly reduced.

In summary, the in vivo experimental results show that, when the LIPUS of the present disclosure is adopted for a treatment, the retinal deposits of mice are significantly reduced, the retina thickness is significantly increased, the visual function is significantly improved, the apoptotic and senescent cells are reduced, and the deposition of lipofuscin is improved. In vitro experimental results show that the oxidative stress degree and the cell senescence both are improved. It can be seen that the LIPUS plays a specified role in the treatment of dAMD.

The above are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for treating dry age-related macular degeneration (dAMD), comprising contacting a product loaded with low-intensity pulsed ultrasound (LIPUS) to an eyeball.

2. The method according to claim 1, wherein the LIPUS has an intensity of 50 mW/cm² to 100 mW/cm² for treatment.

3. The method according to claim 1, wherein the LIPUS has a duration of 10 min to 20 min for treatment.

4. The method according to claim 1, wherein a stimulus-pulse duration is 0.5 s, an inter-interval time is 2 s, and a fundamental frequency is 0.5 MHz.

5. The method according to claim 1, wherein before a treatment with the LIPUS, a medical coupling agent is applied, and then a LIPUS treatment probe is allowed to contact with an eyeball surface for ultrasound treatment.

6. The method according to claim 1, wherein the product comprises a device.
